(19) United States
(12) Patent Application Publication
(10) Pub. No.: US 2007/0156022 A1
(43) Pub. Date: Jul. 5, 2007

(54) Illuminated Medical Devices

(76) Inventor: Rajeev J. Patel, Plano, TX (US)

Correspondence Address: SMITH IP SERVICES, P.C., P.O. Box 997, Rockwall, TX, 75087, US

(21) Appl. No.: 11/686,873

(22) Filed: Mar. 15, 2007

Related U.S. Application Data
(63) Continuation-in-part of application No. 11/031,636, filed on Jan. 7, 2005.
(60) Provisional application No. 60/789,103, filed on Apr. 4, 2006.
(60) Provisional application No. 60/537,150, filed on Jan. 16, 2004.

(51) Int. Cl. A61B 1/267; A61B 1/32

(52) U.S. Cl. 600/199; 600/223

(57) ABSTRACT

Illuminated medical devices. A medical device includes a light source with an outer housing, at least one light bulb, and at least one battery. The light bulb and battery are contained by the outer housing to thereby form a self-contained unit as part of the medical device to illuminate an area in a medical procedure. Another medical device includes a light source with at least one light bulb and at least one battery. The light source is a self-contained unit attached to the medical device and positioned to illuminate an area in a medical procedure. The light source is free of any electrical conductors in an electrical circuit between the battery and integral leads of the light bulb.

### CROSS-REFERENCE TO RELATED APPLICATIONS

The present application claims the benefit of the filing date of provisional U.S. patent application No. 60/789,103 filed Apr. 4, 2006. The present application is also a continuation-in-part of U.S. patent application Ser. No. 11/031,636 filed Jan. 7, 2005 and which claims the benefit of the filing date of provisional U.S. patent application No. 60/537,150 filed Jan. 16, 2004. The entire disclosures of these provisional and non-provisional applications are incorporated herein by this reference.

### BACKGROUND

The present invention relates generally to equipment and methods utilized in medical procedures and, in an embodiment described herein, more particularly provides illuminated medical devices.

In the process of inserting a tube into the trachea of a patient, a device known as a laryngoscope is typically used to aid in opening up a passage for the tube and observing the passage. Unfortunately, most existing laryngoscopes and methods for using the laryngoscopes are deficient in several aspects.

For example, some laryngoscopes visually obstruct the passage, thereby making it difficult to observe the passage, so that the medical personnel intubating the patient cannot tell whether the passage is clear, aligned, or otherwise ready for inserting an intubation tube in the trachea. Some laryngoscopes are provided with a light source to aid in illuminating the passage, but typically these light sources include battery-powered incandescent bulbs and are not of disposable design, so that the batteries and bulb must be periodically replaced and the laryngoscope must be sterilized after each use.

Some laryngoscopes do not adequately align the passage for the tube, thereby making it difficult to insert the tube into the trachea. One typical deficiency in this respect is a failure to maintain control of the tongue. One result of this can be subluxation of the tongue into the pharyngeal cavity, obstructing the view or the passage for the tube into the tracheal ostea. Another deficiency is the lack of manipulation of the tongue in the oral cavity by other laryngoscopes which can obstruct the view of the tracheal ostea.

Therefore, it may be seen that improvements are needed in the art of medical devices and associated methods. These improvements may be useful in medical devices other than laryngoscopes. It is an object of the present invention to provide such improvements.

### SUMMARY

In carrying out the principles of the present invention, medical devices are provided which solve at least one problem in the art. An example is described below in which a laryngoscope is a disposable unit having an integrally-formed handle and blade, and a self-contained light source. Other examples are provided in which various medical devices include a light source having a substantially reduced number of components.

In one aspect of the invention, a medical device is provided which includes a light source having an outer housing, at least one light bulb, and at least one battery. The light bulb and battery are contained by the outer housing to thereby form a self-contained unit as part of the medical device to illuminate an area in a medical procedure.

In another aspect of the invention, a medical device is provided which includes a light source having at least one light bulb and at least one battery. The light source is a self-contained unit attached to the medical device and positioned to illuminate an area in a medical procedure. The light source is free of any electrical conductors in an electrical circuit between the battery and integral leads of the light bulb.

These and other features, advantages, benefits and objects of the present invention will become apparent to one of ordinary skill in the art upon careful consideration of the detailed description of representative embodiments of the invention hereinbelow and the accompanying drawings.

### BRIEF DESCRIPTION OF THE DRAWINGS

FIG. 1 is a cross-sectional view of a method of intubating a patient, the method embodying principles of the present invention;

FIG. 2 is an enlarged scale side elevational view of a laryngoscope which may be used in the method of FIG. 1, the laryngoscope embodying principles of the present invention;

FIG. 3 is an end elevational view of the laryngoscope of FIG. 2;

FIG. 4 is an enlarged scale view of a light source of the laryngoscope of FIG. 2;

FIG. 5 is a side elevational view of another laryngoscope which embodies principles of the present invention;

FIG. 6 is an enlarged scale cross-sectional view of a light source of the laryngoscope of FIG. 5;

FIG. 7 is a cross-sectional view of a vaginal speculum which embodies principles of the present invention;

FIG. 8 is a perspective view of medical loupes which embody principles of the present invention;

FIG. 9 is a side elevational view of a light assembly which embodies principles of the invention; and

FIG. 10 is a side elevational view of another light assembly which embodies principles of the invention.

### DETAILED DESCRIPTION

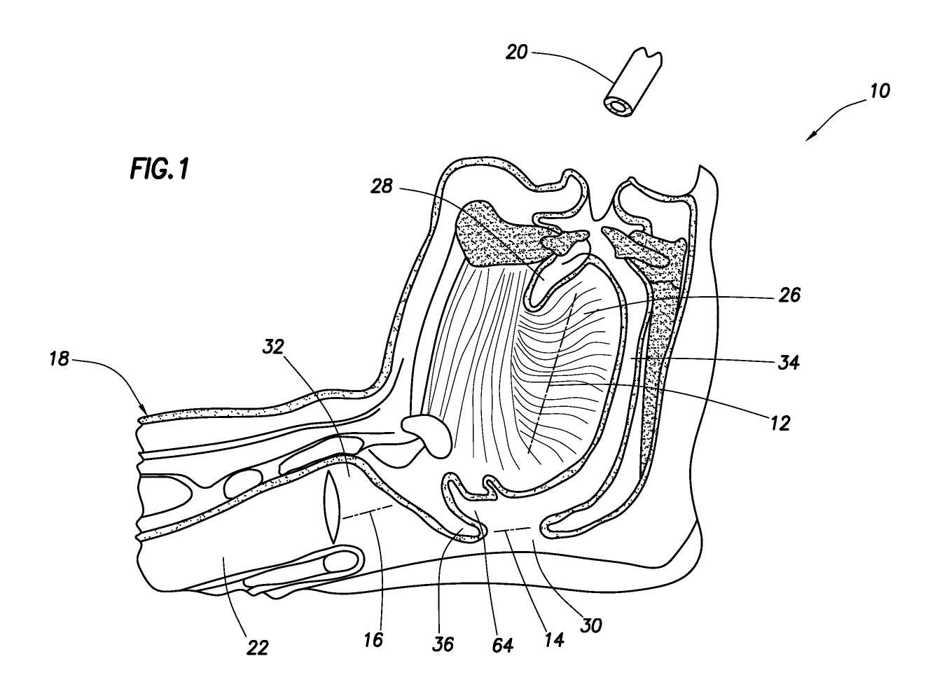

Representatively illustrated in FIG. 1 is a patient intubating method 10 which embodies principles of the present invention. In the following description of the method 10 and other apparatus and methods described herein, directional terms, such as “above”, “below”, “upper”, “lower”, etc., are used for convenience in referring to the accompanying drawings. Additionally, it is to be understood that the various embodiments of the present invention described herein may be utilized in various orientations, such as inclined, inverted, horizontal, vertical, etc., and in various configurations, without departing from the principles of the present invention. The embodiments are described merely as examples of useful applications of the principles of the invention, which is not limited to any specific details of these embodiments.

In the method 10, three anatomical axes 12, 14, 16 of a patient 18 are aligned to facilitate insertion of a tube 20 into a trachea 22 of the patient. The axis 12 is the oral axis extending from the mouth opening to the pharynx along the oral cavity 34. The axis 14 is the pharyngeal axis extending along the pharyngeal cavity 30. The axis 16 is the laryngeal axis extending along the laryngeal cavity 32.

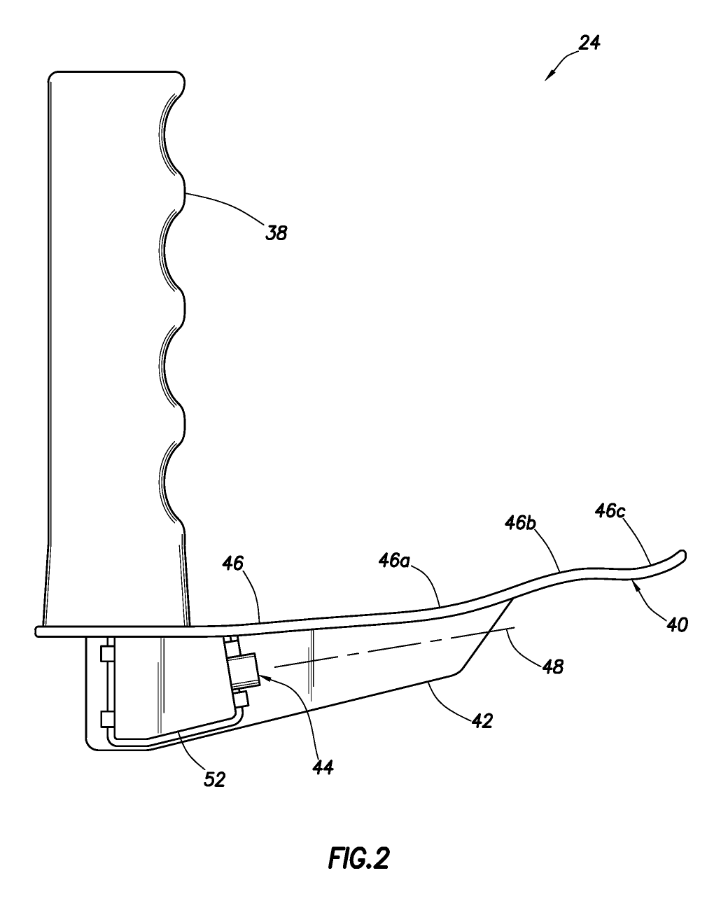

A laryngoscope 24 (shown in FIGS. 2-4) of the invention functions to better align the axes 12, 14, 16. The laryngoscope 24 beneficially displaces the tongue 26 into the submental space 28 and maintains control of the tongue, preventing subluxation of the tongue into the pharyngeal cavity 30. The soft tissue of the pharynx is also displaced by the laryngoscope 24 to permit enhanced observation of the tracheal lumen. A tip of a blade of the laryngoscope 24 may rest in the vallecular area 64, or it may be used to lift the epiglottis 36 if needed to permit enhanced observation of the tracheal lumen.

A light source of the laryngoscope 24 is conveniently positioned to illuminate the passage for the tube 20. The laryngoscope 24 is preferably constructed of clear or translucent materials (such as polycarbonate, etc.) to further enhance the illumination of the passage. In addition, a blade of the laryngoscope 24 may be clear to permit viewing the epiglottis 36 through the blade.

Referring additionally now to FIG. 2, an enlarged scale side view of the laryngoscope 24 is representatively illustrated. In this view it may be seen that the laryngoscope 24 includes a handle 38, a blade 40, a supporting rib 42 and a light source 44. In use, the handle 38 is grasped by a user and an upper surface 46 of the blade 40 is pressed against the tongue 26 and possibly epiglottis 36 of the patient 18 as described in further detail below, to thereby facilitate aligning the three axes 12, 14, 16 in the method 10.

The surface 46 of the blade 40 preferably includes three curved sections 46a, 46b, 46c. The section 46a is concave facing the tongue 26 of the patient 18. This shape allows the tongue 26 to be displaced into the submental space 28 while maintaining control of the tongue (i.e., the tongue will be naturally biased toward the concave depression formed by the surface 46a).

The section 46b is convex facing the tongue 26 of the patient 18. When appropriately positioned in the oral cavity 34, the section 46b will be located at the rear of the tongue 26 and, when pressed against the tongue, will function to prevent subluxation of the tongue into the pharyngeal cavity 30. This portion of the blade 40 also outwardly displaces the soft tissue bounding the pharyngeal cavity 30, permitting better observation of the tracheal lumen.

The section 46c is concave facing the tongue 26 of the patient 18. This section 46c may be used to lift the epiglottis 36 to better observe the tracheal lumen. Alternatively, this portion of the blade 40 may rest in the vallecular area 64 at the rear of the tongue 26.

The rib 42 supports the blade 40 as it is pressed against the tongue 26. Note that the rib 42 extends from the handle 38 portion to a position between the two sections 46b, 46c on the blade 40. This provides substantial support to the portion of the blade 40 having the section 46a thereon which is pressed directly against the tongue 26.

The light source 44 is attached to an inner side of the rib 42. The light source 44 is not obstructed by the blade 40, and can direct light along an axis 48 which is aligned with the anatomical axes 12, 14, 16. If the blade 40 is made of a transparent material, this illumination may also be used to observe the tongue 26 and epiglottis 36 through the blade.

Preferably, the handle 38, blade 40 and rib 42 are integrally constructed of a single piece of material and are disposable, being used only once. One advantage to the laryngoscope 24 being disposable is that the time and expense of sterilizing the laryngoscope after each use is eliminated. However, it should be clearly understood that it is not necessary for the laryngoscope 24 to be disposable. The laryngoscope 24 could instead be reusable, in which case the integral construction of the handle 38, blade 40 and rib 42 will eliminate the possibility that the blade might be sterilized without the handle also being sterilized (e.g., as could happen with prior non-integrally constructed laryngoscopes).

If the laryngoscope 24 is disposable, the light source 44 is also preferably a self-contained unit in which no replacement of batteries or bulb is provided for. However, many alternatives can be used for the light source 44. If the laryngoscope 24 is reusable, then the batteries and bulb could be conveniently replaceable. As another alternative, the light source 44 could have the batteries positioned in the handle 38, with the bulb either in the handle or attached to the rib 42.

If the bulb is in the handle 38, a fiber optic line or other light conduit may be used to direct the light from the handle to a desired position on or adjacent the rib 42. Light may also be transmitted directly by the material of the laryngoscope 24 itself. For example, the blade 40 and/or rib 42 may be made of a transparent or translucent material to transmit light from the light source 44 to illuminate the oral cavity 34, pharyngeal cavity 30 and laryngeal cavity 32.

Referring additionally now to FIG. 3, an end view of the laryngoscope 24 is depicted. In this view it may be seen that the rib 42 is laterally offset from a center axis 50 of the blade 40. This permits enhanced access for inserting the tube 20 while the laryngoscope 24 is in the oral cavity 34.

Note that the light source 44 is positioned toward the blade axis 50 from the rib 42. This permits the light source 44 to effectively illuminate the oral, pharyngeal and laryngeal cavities 34, 30, 32 without obstruction from the rib 42. However, if the rib 42 is made of a transparent or translucent material, light may be transmitted from the light source 44 through the rib.

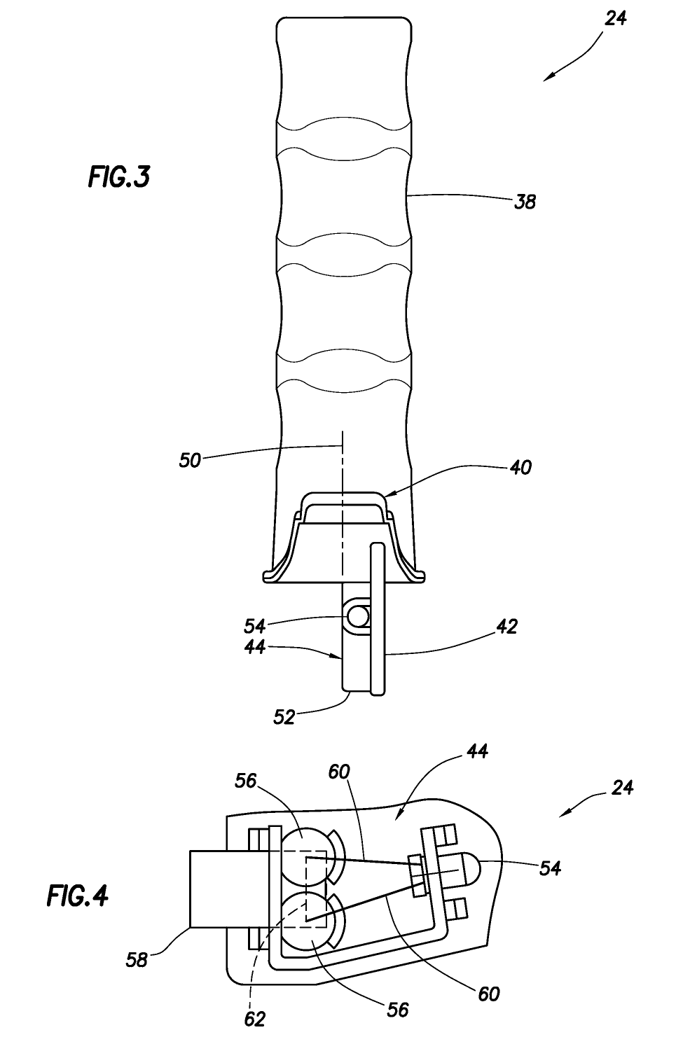

Referring additionally now to FIG. 4, an enlarged side view of the light source 44 is shown with a cover 52 removed. In this view it may be seen that the light source 44 includes a bulb 54 and batteries 56.

The bulb 54 is preferably a high intensity light emitting diode (LED) which consumes relatively little electrical power. Other types of illuminating bulbs (such as incandescent, fluorescent, etc.) may be used in place of the bulb 54, if desired. In addition, any number of bulbs 54 may be used.

A switch in the form of a small strip of insulating material 58 is used to prevent the bulb 54 from illuminating until just prior to using the laryngoscope 24. The material 58 is withdrawn from the light source 44, thereby allowing wire leads 60 of the bulb 54 to contact the batteries 56 and thus supply electrical power to the bulb. A wire contact 62 is used to electrically connect opposite sides of the batteries 56.

Note that, in this disposable embodiment of the laryngoscope 24, the light source 44 cannot be turned off. After withdrawing the material 58, the bulb 54 will continue to illuminate until the batteries 56 are discharged. However, if the laryngoscope 24 is to be reused, a switch which may be turned off can be used in place of the material 58.

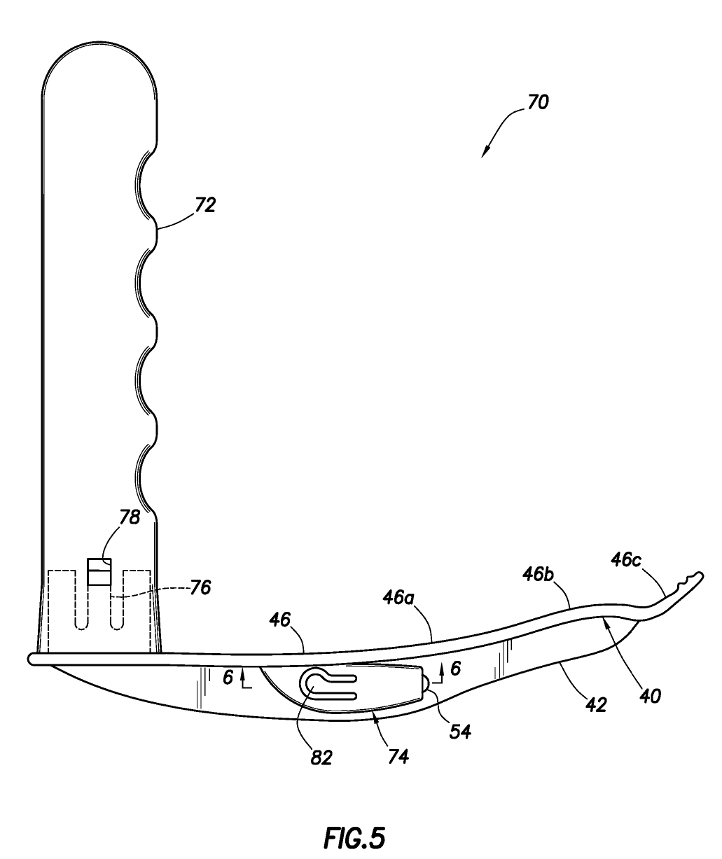

Referring additionally now to FIG. 5, an alternate configuration of a laryngoscope 70 which embodies principles of the present invention is representatively illustrated. The laryngoscope 70 of FIG. 5 is similar in many respects to the laryngoscope 24 described above, but is also different in several respects. For example, the blade 40 is separately formed from a handle 72, and a different light source 74 is used, in the laryngoscope 70 of FIG. 5.

Separate forming of the blade 40 and handle 72 permits more convenient and efficient distribution and storage of the laryngoscope 70. Just prior to use, the handle 72 is snapped onto the blade 40, so that locking tabs 76 securely engage recesses 78. If the laryngoscope 70 is designed for only a single use, this attachment between the blade 40 and handle 72 may not be releasable, but if multiple uses are contemplated, the handle 72 may be releasable from the blade 40 to allow for convenient and thorough sterilization after each use.

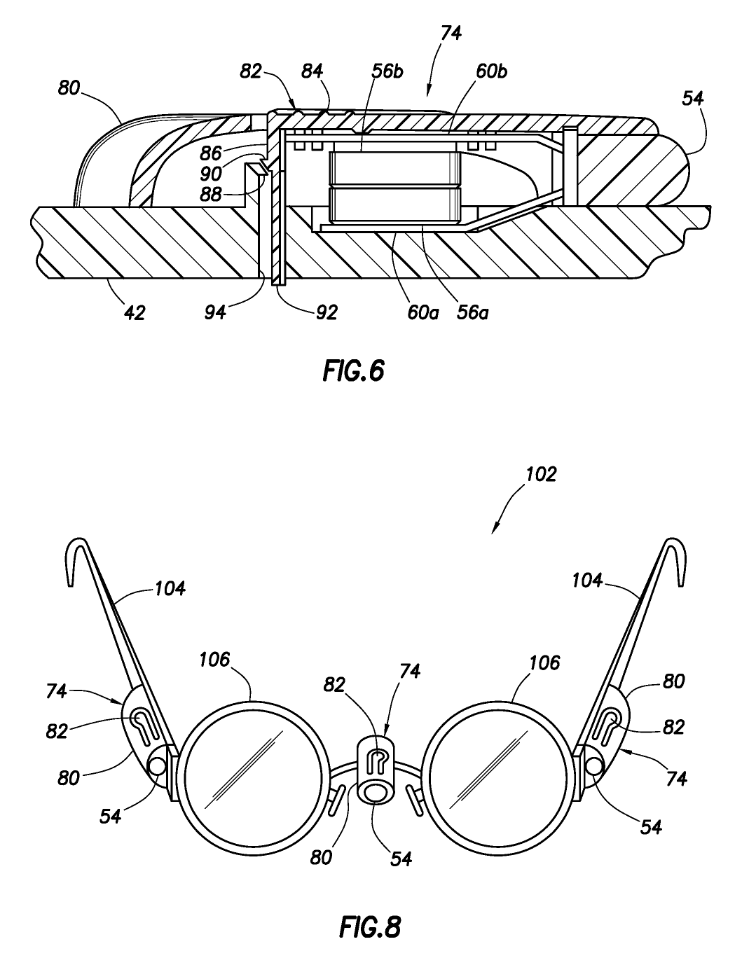

The light source 74 is preferably completely self-contained, including both the bulb 54 and batteries 56a, b in a single enclosure. A cross-sectional view of the light source 74 is representatively illustrated in FIG. 6. In this view it may be seen that the light source 74 differs substantially from the light source 44 described above.

One significant difference is that the batteries 56a, b are stacked (one on top of the other) between the rib 42 and an outer housing 80 of the light source 74. Another significant difference is that the integral leads 60 of the bulb 54 straddle the batteries 56a, b. Yet another difference is that the light source 74 includes a switch 82 to turn on the light source, with the switch being preferably integrally formed with the outer housing 80 and bulb leads 60, and without requiring any additional conductive elements. It will be appreciated that these differences substantially improve the performance, economy and convenience of using the laryngoscope 70.

The switch 82 includes an elongated finger or tab 84 integrally formed as a single piece with the outer housing 80. The tab 84, in turn, has a collet-type finger 86 integrally formed thereon which is shaped to releasably engage a shoulder 88 integrally formed on the rib 42.

When the tab 84 is depressed inwardly relative to the outer housing 80 (downwardly as viewed in FIG. 6), a projection 90 on the finger 86 will engage the shoulder 88 and releasably retain the tab in its depressed position. In this position, an elongated release member 92 will project outwardly through an opening 94 formed in the rib 42.

The tab 84 may be released to spring back to its original position relative to the housing 80 by applying a sufficient force to the release member 92 to thereby disengage the projection 90 from the shoulder 88. Note that the member 92 is preferably integrally formed with the tab 84 and finger 86.

One of the bulb leads 60a is retained between one of the batteries 56a and the rib 42. This provides electrical contact between the lead 60a and the battery 56a. The other bulb lead 60b is attached to the tab 84, so that the bulb lead displaces with the tab.

When the tab 84 is in its released position as depicted in FIG. 6, the lead 60b is maintained separated from the battery 56b. However, when the tab 84 is depressed inwardly to its depressed position, the lead 60b contacts the battery 56b, and an electrical circuit is formed, thereby lighting the bulb 54.

Note that no additional electrical conductors are required to complete the electrical circuit between the bulb 54 and the batteries 56a, b. This reduces the number of parts required to construct the light source 74, reduces the possibility of failure, and reduces the cost of manufacturing the light source.

Although two batteries 56a, b are illustrated, any number of batteries (including one) may be used in keeping with the principles of the invention. Although only one bulb 54 is illustrated, multiple bulbs may be used in keeping with the principles of the invention.

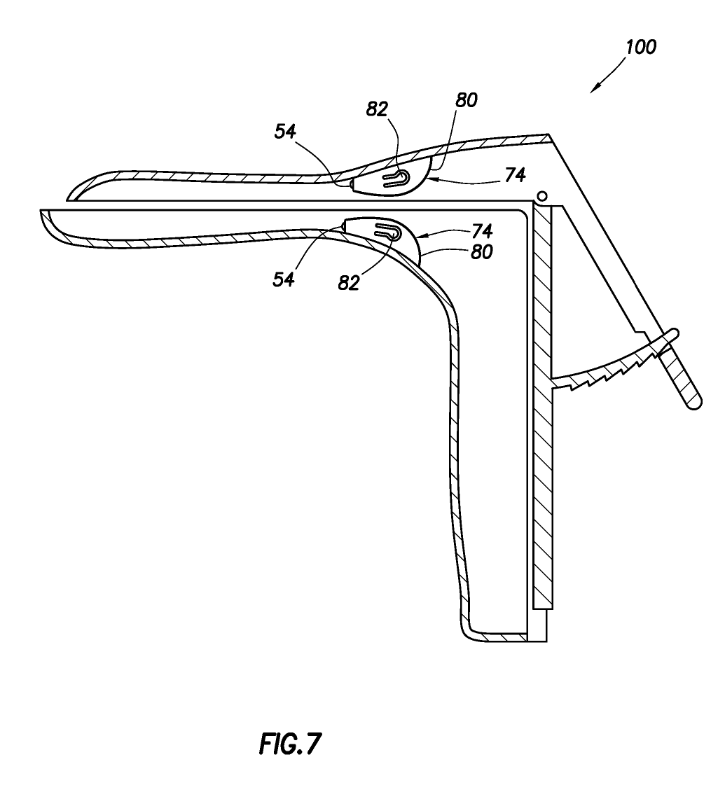

Referring additionally now to FIG. 7, a cross-sectional view of a vaginal speculum 100 embodying principles of the present invention is representatively illustrated. The speculum 100 demonstrates that various types of medical devices, other than laryngoscopes, may benefit from the principles of the invention.

The speculum 100 as depicted in FIG. 7 includes two of the light sources 74 on interior surfaces of the speculum. Only one of the light sources 74 would typically be used. It will be appreciated that the light sources 74 provide enhanced illumination of the vaginal walls and cervix in use.

Although the switch 82 is depicted in FIG. 7 as being positioned facing laterally with respect to the outer housing 80 for illustrative clarity, other positions of the switch may be used in keeping with the principles of the invention.

Referring additionally now to FIG. 8, another type of medical device which utilizes the light source 74 is representatively illustrated. In FIG. 8, the medical device is of the type known as medical or surgical loupes 102.

Two light sources 74 are positioned on respective earpieces 104 of the loupes 102. Another light source 74 is positioned laterally between lenses 106 of the loupes 102. Typically, only the light source 74 between the lenses 106, or the two light sources on the earpieces 104 would be used, but it should be understood that any number, combination and positioning of light sources may be used in keeping with the principles of the invention.

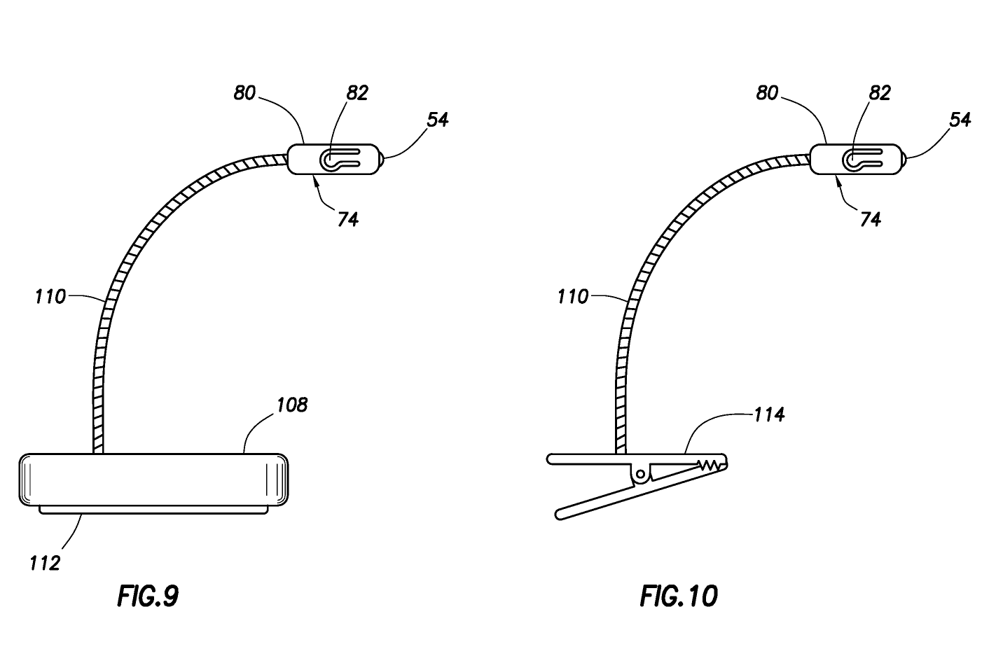

Referring additionally now to FIG. 9, the light source 74 is representatively illustrated attached to a base 108 via a flexible arm 110. This allows the light source 74 to be conveniently positioned proximate an area requiring illumination in a variety of medical procedures.

The base 108 may be relatively heavy to thereby provide a stable platform for the arm 110 and light source 74. Alternatively, or in addition, the base 108 could be magnetic or have a pad 112 on a bottom surface thereof. The pad 112 could be magnetic, or could have an adhesive for securing the base 108, either permanently or temporarily, to a convenient surface.

The base 108, or at least a portion thereof, could be flexible to allow it to conform to irregular surfaces.

Referring additionally now to FIG. 10, the light source 74 is representatively illustrated attached to a clamp 114 via the arm 110. Alternatively, the light source 74 could be attached directly to the clamp 114, without use of the arm 110.

The clamp 114 permits the light source 74 to be secured to a variety of objects to thereby provide convenient illumination in a variety of medical procedures.

In each of the embodiments described above, it should be understood that any shape, size, number, position, etc. of the light sources 44, 74 may be used in keeping with the principles of the invention.

Of course, a person skilled in the art would, upon a careful consideration of the above description of representative embodiments of the invention, readily appreciate that many modifications, additions, substitutions, deletions, and other changes may be made to these specific embodiments, and such changes are within the scope of the principles of the present invention. Accordingly, the foregoing detailed description is to be clearly understood as being given by way of illustration and example only, the spirit and scope of the present invention being limited solely by the appended claims and their equivalents.

What is claimed is:

1. A medical device, comprising:
a light source including at least one light bulb and at least one battery, the light source being a self-contained unit attached to the medical device and positioned to illuminate an area in a medical procedure; and
the light source being free of any electrical conductors in an electrical circuit between the at least one battery and integral leads of the at least one light bulb.

2. The medical device of claim 1, wherein the at least one battery includes multiple batteries, and wherein the light source is free of any electrical conductors in the electrical circuit between the batteries.

3. The medical device of claim 1, wherein the light source further includes a switch integrally formed with an outer housing and an integral lead of the at least one light bulb.

4. The medical device of claim 3, wherein the switch includes a tab displaceable relative to the outer housing and integrally formed as a single piece with the outer housing.

5. The medical device of claim 4, wherein the light bulb lead is attached to the tab for displacement relative to the outer housing with the tab.

6. The medical device of claim 4, wherein displacement of the tab relative to the housing causes the light bulb lead to contact the battery to thereby form the electrical circuit.

7. The medical device of claim 1, wherein the medical device is a laryngoscope, and wherein the light source is attached to a rib supporting a blade of the laryngoscope.

8. The medical device of claim 1, wherein the medical device is a vaginal speculum, and wherein the light source is attached to an interior surface of the speculum.

9. The medical device of claim 1, wherein the medical device is a set of loupes, and wherein the light source is attached to an earpiece of the loupes.

10. The medical device of claim 1, wherein the medical device is a set of loupes, and wherein the light source is positioned between lenses of the loupes.

11. The medical device of claim 1, wherein the light source is attached to at least one of a base and a clamp.

12. A medical device, comprising:
a light source including an outer housing, at least one light bulb, and at least one battery; and
the light bulb and battery being contained by the outer housing to thereby form a self-contained unit as part of the medical device to illuminate an area in a medical procedure.

13. The medical device of claim 12, wherein the light source is free of any electrical conductors in an electrical circuit between the at least one battery and integral leads of the light bulb.

14. The medical device of claim 12, wherein the at least one battery includes multiple batteries, and wherein the light source is free of any electrical conductors in the electrical circuit between the batteries.

15. The medical device of claim 12, wherein the light source further includes a switch integrally formed with the outer housing and an integral lead of the light bulb.

16. The medical device of claim 15, wherein the switch includes a tab displaceable relative to the outer housing and integrally formed as a single piece with the outer housing.

17. The medical device of claim 16, wherein the light bulb lead is attached to the tab for displacement relative to the outer housing with the tab.

18. The medical device of claim 16, wherein displacement of the tab relative to the housing causes the light bulb lead to contact the battery to thereby form the electrical circuit.

19. The medical device of claim 12, wherein the medical device is a laryngoscope, and wherein the light source is attached to a rib supporting a blade of the laryngoscope.

20. The medical device of claim 12, wherein the medical device is a vaginal speculum, and wherein the light source is attached to an interior surface of the speculum.

21. The medical device of claim 12, wherein the medical device is a set of loupes, and wherein the light source is attached to an earpiece of the loupes.

22. The medical device of claim 12, wherein the medical device is a set of loupes, and wherein the light source is positioned between lenses of the loupes.

23. The medical device of claim 12, wherein the light source is attached to at least one of a base and a clamp.
